(19) United States
(12) Patent Application Publication
(10) Pub. No.: US 2020/0384138 A1
(43) Pub. Date: Dec. 10, 2020

(54) SYSTEMS AND METHODS FOR DISINFECTING A FACILITY

(71) Applicant: Barry Hunt, Cambridge (CA)

(72) Inventor: Barry Hunt, Cambridge (CA)

(21) Appl. No.: 16/869,065

(22) Filed: May 7, 2020

Related U.S. Application Data
(60) Provisional application No. 62/844,475, filed on May 7, 2019.

(51) Int. Cl. A61L 2/10; A61L 2/26; A61L 2/24

(52) U.S. Cl. CPC A61L 2/10; A61L 2/26; A61L 2202/14; A61L 2202/11; A61L 2/24

(57) ABSTRACT

Systems and methods of disinfection a facility are described herein. In at least one embodiment, the systems include a thermal-based imaging sensor configured to detect thermal infrared radiation in the facility, the facility having a plurality of regions; an ultraviolet light source to provide ultraviolet radiation to disinfect the facility; and a controller configured to: receive an input signal from the thermal-based sensor, the input signal comprising temperature data based on the detected thermal infrared radiation in the facility; determine a presence or an absence of a person in one of the plurality of regions of the facility based on the temperature data; and upon determining the absence of a person in the one of the plurality of regions of the facility, activate the ultraviolet light source to disinfect the one of the plurality of regions.

### CROSS-REFERENCE TO RELATED APPLICATIONS

This application claims the benefit of U.S. Provisional Patent Application No. 62/844,475 that was filed on 7 May 2019, the contents of which are incorporated herein by reference for all purposes.

### TECHNICAL FIELD

The embodiments disclosed herein relate to systems and methods for sterilization and disinfection and, in particular to systems and methods for targeted automatic disinfection of a facility.

### BACKGROUND

Ultraviolet (UV) radiation is commonly used for disinfecting and/or sterilizing surfaces in facilities to eliminate microorganisms that cause infections and thereby reduce the transmission of infections. UV radiation is particularly useful for disinfecting surfaces in facilities where antibiotic resistant organisms are present, such as in hospitals and other medical clinics.

UV radiation decontamination systems sterilize surfaces by exposing them to a sufficient intensity of UV radiation for a sufficient exposure period to destroy the microorganisms thereon. Exposure to UV radiation can have a harmful effect on humans, so in order to disinfect a facility with UV radiation people must leave the facility. This disruption can be inconvenient.

Accordingly, there is a need for new or improved systems and methods for disinfecting facilities.

### SUMMARY

According to some embodiments, a facility disinfection system is described herein. The system includes a thermal-based imaging sensor configured to detect thermal infrared radiation in the facility, the facility having a plurality of regions; an ultraviolet light source to provide ultraviolet radiation to disinfect the facility; and a controller configured to: receive an input signal from the thermal-based sensor, the input signal comprising temperature data based on the detected thermal infrared radiation in the facility; determine a presence or an absence of a person in one of the plurality of regions of the facility based on the temperature data; and upon determining the absence of a person in the one of the plurality of regions of the facility, activate the ultraviolet light source to disinfect the one of the plurality of regions.

According to some embodiments, the controller is configured to upon determining the presence of a person in the one of the plurality of regions of the facility, control the ultraviolet light source to inhibit providing the ultraviolet radiation to the one of the plurality of regions, and activate the ultraviolet light source to disinfect a second portion of the facility portion of the facility upon determining an absence of the person in the portion of the facility.

According to some embodiments, the ultraviolet light source is controllable and the controller controls the dissemination of UV radiation produced by the ultraviolet light source to a selected region of the plurality of regions.

According to some embodiments, the ultraviolet light from the ultraviolet light source is collimated to control dissemination.

According to some embodiments, the ultraviolet light source comprises a shield to deflect UV radiation produced by the ultraviolet light source.

According to some embodiments, the controller controls movement of the shield to control the dissemination of UV radiation produced by the ultraviolet light source to a selected region of the plurality of regions.

According to some embodiments, the ultraviolet light from the ultraviolet light source is rotatable.

According to some embodiments, a method of disinfecting a facility is provided herein. The method includes receiving an input signal from a thermal-based sensor, the input signal comprising temperature data based on thermal infrared radiation detected by the sensor in the facility; determining a presence or an absence of a person in one of a plurality of regions of the facility based on the temperature data; and, upon determining the absence of a person in the one of the plurality of regions of the facility, activating the ultraviolet light source to disinfect the one of the plurality of regions.

According to some embodiments, upon determining the presence of a person in the one of the plurality of regions of the facility, the method further includes controlling the ultraviolet light source to inhibit providing the ultraviolet radiation to the one of the plurality of regions, and activating the ultraviolet light source to disinfect a second portion of the facility portion of the facility upon determining an absence of the person in the portion of the facility.

According to some embodiments, a facility disinfection system, is described herein. The system includes at least one sensor configured to detect an object in the facility, track a position of the object in the facility and output object tracking data based on the position of the object over time, the facility having a plurality of regions; an ultraviolet light source to provide ultraviolet radiation to disinfect the facility; and a controller configured to: receive the signal from the at least one sensor, the signal comprising the object tracking data; determine a presence or an absence of a person in one of the plurality of regions of the facility based on the object tracking data; and upon determining the absence of a person in the one of the plurality of regions of the facility, activate the ultraviolet light source to disinfect the one of the plurality of regions.

According to some embodiments, a method of disinfecting a facility is described herein. The method includes: receiving an input signal from a sensor, the input signal comprising object tracking data; determining a presence or an absence of a person in one of a plurality of regions of the facility based on the object tracking data; and upon determining the absence of a person in the one of the plurality of regions of the facility, activating the ultraviolet light source to disinfect the one of the plurality of regions.

According to some embodiments, the at least one sensor is a Light Detection and Ranging (LIDAR) sensor.

According to some embodiments, the object tracking data includes 2D tracking data and/or 3D tracking data.

Other aspects and features will become apparent, to those ordinarily skilled in the art, upon review of the following description of some exemplary embodiments.

### BRIEF DESCRIPTION OF THE DRAWINGS

The drawings included herewith are for illustrating various examples of articles, methods, and apparatuses of the present specification. In the drawings:

FIG. 1 is a block diagram of a facility disinfection system, according to one embodiment;

FIG. 2 is a schematic diagram of sensors and respective regions within a facility of the facility disinfection system of FIG. 1, according to one embodiment;

FIG. 3 is a perspective view of a light source of the facility disinfection system of FIG. 1, according to one embodiment;

FIG. 4 is a perspective view of a light source of the facility disinfection system of FIG. 1 having a shield, according to one embodiment;

FIG. 5 is a schematic diagram of a plurality of lights sources of the facility disinfection system of FIG. 1 arranged in a facility, according to one embodiment;

FIGS. 6A and 6B are a top view and a side view, respectively, of a movable light source of the facility disinfection system of FIG. 1, according to one embodiment; and

FIG. 7 is a method of disinfecting a facility, according to one embodiment.

The skilled person in the art will understand that the drawings, described below, are for illustration purposes only. The drawings are not intended to limit the scope of the applicants' teachings in anyway. Also, it will be appreciated that for simplicity and clarity of illustration, elements shown in the figures have not necessarily been drawn to scale. For example, the dimensions of some of the elements may be exaggerated relative to other elements for clarity. Further, where considered appropriate, reference numerals may be repeated among the figures to indicate corresponding or analogous elements.

### DETAILED DESCRIPTION

Various apparatuses or processes will be described below to provide an example of each claimed embodiment. No embodiment described below limits any claimed embodiment and any claimed embodiment may cover processes or apparatuses that differ from those described below. The claimed embodiments are not limited to apparatuses or processes having all of the features of any one apparatus or process described below or to features common to multiple or all of the apparatuses described below.

Terms of degree such as “about” and “approximately” as used herein mean a reasonable amount of deviation of the modified term such that the end result is not significantly changed. These terms of degree should be construed as including a deviation of at least ±5% or at least ±10% of the modified term if this deviation would not negate the meaning of the word it modifies.

The term “comprising” and its derivatives, as used herein, are intended to be open ended terms that specify the presence of the stated features, elements, components, groups, integers, and/or steps, but do not exclude the presence of other unstated features, elements, components, groups, integers and/or steps. The foregoing also applies to words having similar meanings such as the terms, “including”, “having” and their derivatives.

The term “consisting” and its derivatives, as used herein, are intended to be closed terms that specify the presence of the stated features, elements, components, groups, integers, and/or steps, but exclude the presence of other unstated features, elements, components, groups, integers and/or steps.

The term “consisting essentially of”, as used herein, is intended to specify the presence of the stated features, elements, components, groups, integers, and/or steps as well as those that do not materially affect the basic and novel characteristic(s) of features, elements, components, groups, integers, and/or steps.

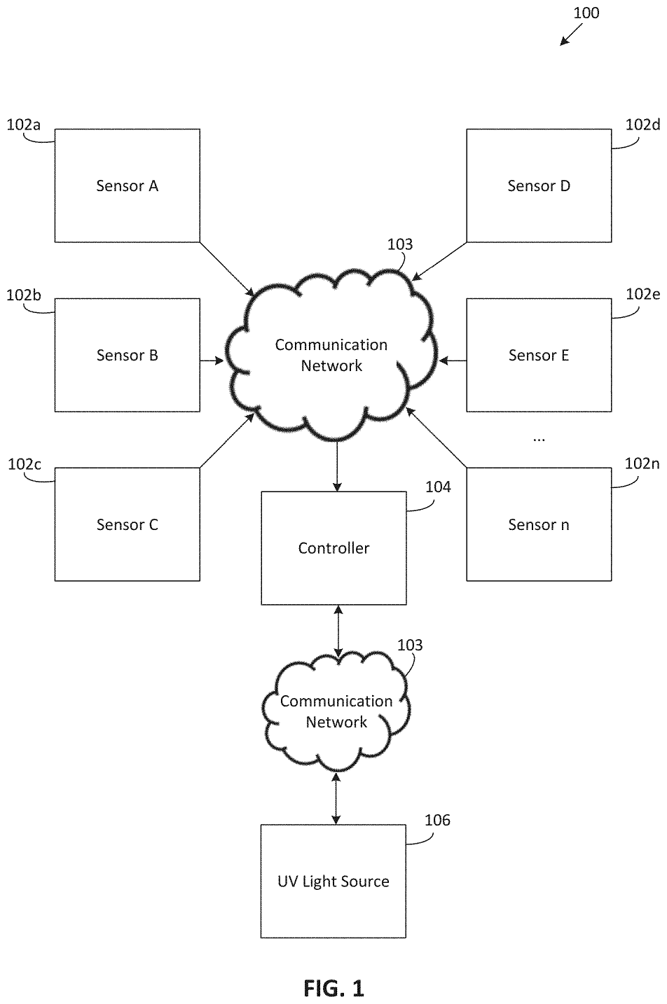

Referring now generally to FIG. 1, illustrated therein is a targeted automatic UV disinfection system 100, according to one exemplary embodiment. The disinfection system 100 generally includes a plurality of sensors 102 (i.e. sensors 102a, 102b, 102c, 102d, 102e, . . . , 102n), a communication network 103, a controller 104 and a light source 106.

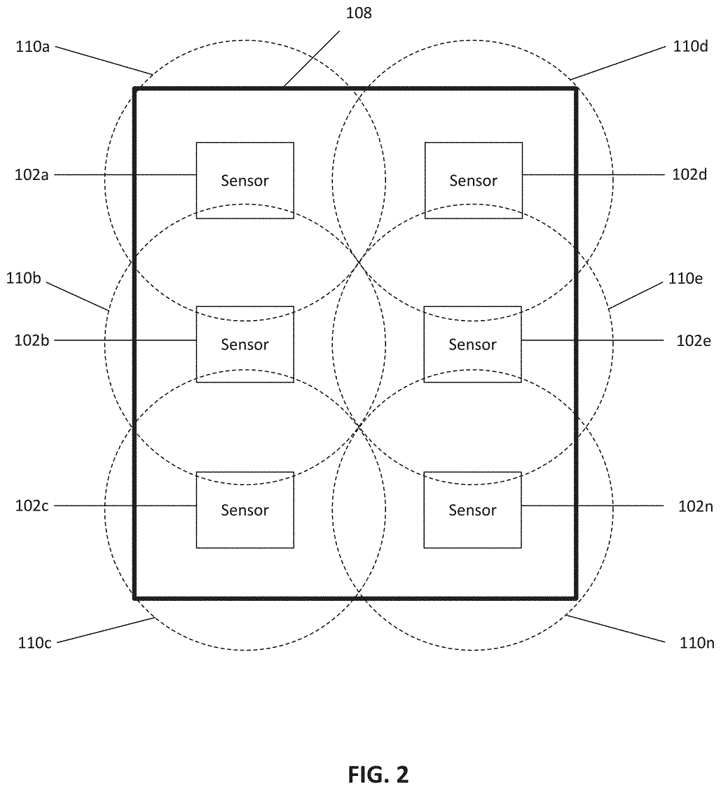

As shown in FIG. 2, sensors 102 generally measure thermal infrared radiation (e.g. as pixel data) emitted in a respective region 110 (i.e. regions 110a, 110b, 110c, 110d, 110e, . . . , 110n) of a facility 108. For example, thermal infrared radiation is generally emitted by objects, including people, in the mid-infrared thermal band (i.e. radiation with a wavelength in a range from about 3 microns to 8 microns) and far-infrared thermal band (i.e., radiation with a wavelength in a range from about 8 microns to 14 microns) of the electromagnetic spectrum.

Sensors 102 detect temperature changes in regions 110 caused by objects emitting radiation in the mid-infrared thermal band and far-infrared thermal band of the electromagnetic spectrum. Slightly shorter or slightly longer ranges may also yield acceptable detection results. Sensors 102 include one or more thermal cameras having pixel arrays sensitive to the mid-infrared and/or far-infrared bands of the electromagnetic spectrum. For example, in one embodiment, sensors 102 may be thermal activity sensors (TASs) manufactured by ULIS, a manufacturer of thermal sensors. TASs are thermal activity sensors that run entirely on battery power. TASs can provide 80x80 pixels thermal activity sensors and transmit occupancy rates at regular intervals (approximately every two minutes), without compromising the privacy of the occupants. In another embodiment, sensors 102 may be thermal activity sensors that do not run on battery power.

Facility 108 can include any number of sensors 102 arranged in any pattern to measure thermal infrared radiation emitted within the facility. For example, in some embodiments, a single sensor 102 can be used to detect thermal infrared radiation in one or more regions 110 of a facility 108. The sensors 102 are preferably arranged in facility 108 so that the area of their respective regions 110 covers the entirety of the facility 108. Accordingly, neighboring respective regions 110 may overlap to provide for detection of thermographic data within the entire area of facility 108. Regions 110 are shown in FIG. 2 as being approximately circular in shape, however the shape and size of regions 110 are defined by sensors 102. Accordingly, regions 110 can be any appropriate shape and size for sensing thermal infrared radiation therein.

In one embodiment, each sensor 102 may detect mid-infrared and/or far-infrared bands of the electromagnetic spectrum for a region approximately 30 square meters in size. Facility 108 can represent a traditional physical facility (e.g. a hospital) or a portion of a traditional physical facility (e.g. a patient room within a hospital).

Thermal infrared radiation measured by sensors 102 of objects in regions 110 is suitable to indicate temperature variations present within regions 110. Thermal infrared radiation measurements can be stored as pixel data by sensors 102 and used to produce a thermogram of the area of region 110 corresponding to each respective sensor 102. For example, sensor 102a can produce a thermogram of infrared radiation measurements for region 110a.

In one embodiment, thermograms can be produced by sensors 102 that are representative of local temperature changes in regions 110 of facility 108 caused by the movement of humans into, out of and/or within facility 108, for example. Thermograms from sensors 102 can be produced over time and can be combined and analyzed together to track changes in thermographic radiation, and therefore movement of persons, in the facility 108 over time. For example, changes in thermographic radiation over time within facility 108 may represent the entrance, movement within (i.e. change in position) and exit of a person through facility 108. For example, changes in temperature in regions 110 of facility 108 that are caused by the presence of a person, and with suitable monitoring of emissions from the person in the thermal infrared spectrum over time within regions 110 of facility 108, movement of the person between regions 110 of the facility 108 can be detected.

Returning to FIG. 1, sensors 102 each include a transmitter (not shown). After measuring thermal infrared radiation and storing pixel data derived therefrom emitted within a respective region 110, each sensor 102 can provide the thermal infrared image data to a controller 104 via its transmitter over a communication network 103.

Controller 104 receives the thermal infrared image data transmitted by each of the sensors 102 in facility 108 over communication network 103. Controller 104 may store the thermal infrared image data in storage (not shown) or may transmit the thermal infrared image data to a server (not shown) for storage, also over communication network 103.

It should be noted that two exemplary communications networks 103 are shown in FIG. 1. The two communication networks 103 shown in FIG. 1 can represent a single communication network 103, two individual communication networks 103 of the same type (e.g. WiFi networks) or two individual communication networks 103 of different types (e.g. a Bluetooth® network and a WiFi network). Exemplary communication networks 103 include local area networks (LANs) and/or wide area networks (WANs). Such networking environments are commonplace in offices, enterprise-wide computer networks, intranets, and the Internet. Further, communication networks 103 may include other forms of wireless communication including but not limited to RFID and Bluetooth. For instance, each sensor 102 may transmit thermal infrared image data to controller 104 over one type of communication network (e.g. Bluetooth) and controller 104 may transmit a signal to a UV light source 106 over another type of communication network (e.g. Internet). As noted above, controller 104 may also transmit thermal infrared image data received from the sensors 102 to a server (not shown).

When utilized in a WAN networking environment, controller 104 might comprise a modem or other means for establishing communications over the WAN, such as the Internet. It will be appreciated by those of ordinary skill in the art that the network connections shown are exemplary and other means of establishing a communications link between each sensor 102 and the controller 104 and between the controller 104 and the UV light source 106 might be utilized.

Controller 104 is connected to the UV light source 106 by a UV light source hardware interface 151. Controller 104 is connected to the sensors 102 by a sensor interface 152. In some embodiments, controller 104 is located apart from UV light source 106 and sensors 102. Controller 104 is connected to UV light source 106 and sensors 102 via the respective interfaces and suitable circuitry (e.g. communication network 103 and/or wires).

Controller 104 may be implemented in hardware, software, firmware, or combinations thereof. Controller 104 may include a processing element coupled with a memory element that in combination are able to execute software code segments that implement the control function. Controller 104 may also include microcomputers, microprocessors, microcontrollers, programmable intelligent computers (PICs), field-programmable gate arrays (FPGAs), programmable logic devices (PLDs), programmable logic controllers (PLCs), and the like. The controller 26 may also be formed or created from one or more code segments of a hardware description language (HDL). Controller 104 may also include a memory component such as hard-disk drives, optical disks, floppy disks, random-access memory (RAM), read-only memory (ROM), cache memory, programmable ROM (PROM), erasable PROM (EPROM), and the like. In addition, controller 104 may include other data input devices such as keyboards, keypads, mice or other pointing devices, knobs, buttons, switches, and the like. The controller 104 may include other data output devices such as screens, monitors, displays, speakers, LEDs, liquid crystal displays (“LCDs”), and the like. Furthermore, the controller 104 may include data interfaces, such as a computer network interface, to allow the system 100 to send and receive data from other computers, networks, or systems. Additionally, the controller may be operable with a mainframe controller for the building in which the elevator is housed.

Controller 104 may also include one or more timing elements. The timing elements may include count-down timers that wait for a predetermined amount of time, and count-up timers that measure the duration of an event.

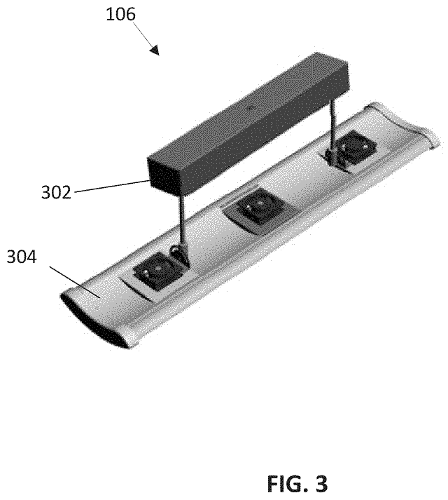

Referring now to FIG. 3, one embodiment of UV light source 106 is shown therein. UV light source 106 generally provides a source of disinfecting radiation in the UV radiation range (e.g. having a wavelength in a range between about 100 nm and 400 nm). It is known that peak effectiveness for UV radiation as a germicide or disinfectant has a wavelength is in a range of about 240 nm to about 280 nm, known as UV-C radiation. UV radiation between having a wavelength in this range 000 may destroy DNA in living microorganisms and break down organic material found in the air in an indoor environment. The wavelength of UV light source 106 is generally fixed when the source is manufactured, although in some embodiments, the wavelength of UV light source 106 may be varied after installation, or during operation.

UV light source 106 includes one or more components operable to emit UV radiation such as lasers, electric arc lamps, pressurized mercury bulbs, or the like. Typically, UV light source 106 includes one or more tube-shaped bulbs (not shown) to provide UV radiation.

Generally, as shown in FIG. 3, UV light source 106 comprises a ballast 302 coupled to a body 304 housing at least one UV light bulb (not shown) or other appropriate mechanism for emitting UV radiation. Body 304 further comprises electrical hardware for electrically powering the UV bulb(s). Body 304 may have a rectangular shape (as shown) of a size appropriate to accommodate one or more of the UV light bulbs. Body 304 may also have a circular shape (e.g. as a potlight) or any other appropriate shape to house a UV radiation emitting mechanism (e.g. a bulb). Body 304 may be manufactured from metals, plastics, wood, or other suitable materials. Body 304 may also comprise a reflective surface (not shown) directly behind the UV light bulb such that any UV radiation incident to the reflective surface is reflected off of the surface and into facility 108. Additionally, a fan may optionally be positioned within or adjacent to body 304 to provide air circulation to cool the UV light bulb. In the embodiment shown in FIG. 3, UV light source 106 is used as a ceiling mounted light source where ballast 302 is mounted to or within a ceiling (not shown) and body 304 is coupled to ballast 402. It should be understood that UV light source 106 can be mounted to any appropriate partition (e.g. a wall or floor) within a facility 108 (e.g. a room).

Operation of the UV light source 106 may be controlled by the controller 104, such that the controller 104 sends a signal to the UV light source 106 to control activation and/or deactivation of UV light source 106, for example. UV light source 106 may also send a signal to the controller 104 regarding its status, for example, whether the UV light source 106 is on or off.

In one embodiment, the UV light source 106 is powered at all times. In this embodiment, the UV light source 106 may be positioned within a partition to be shielded so UV radiation emitted therefrom does not enter facility 108. In another embodiment, UV light source 106 can be powered on and off and the controller 104 is operable to control powering the UV light source 106 on and off.

Generally, UV light source 106 is controlled by controller 104 to provide UV radiation to selectable regions 110 of facility 108 to inhibit people in facility 108 from being exposed to UV radiation.

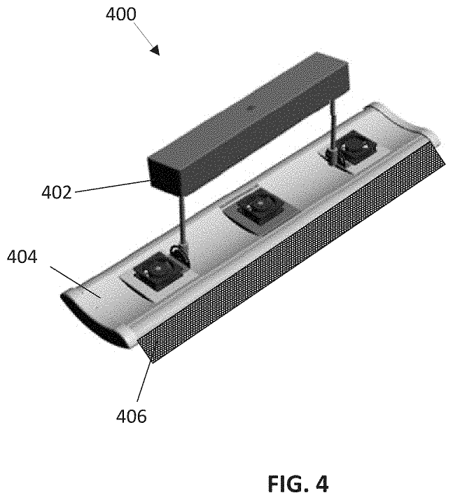

For example, in one embodiment illustrated in FIG. 4, a UV light source 400 comprising a ballast 402 coupled to a body 404 housing at least one bulb (not shown) is provided. In this embodiment, UV light source 400 comprises a blind and/or shield 406 to block and/or reflect UV radiation emitted from the bulb therein to inhibit a person in facility 108 from be exposed to UV radiation (e.g. to inhibit UV radiation from directly contacting a person or people present in facility 108) when the UV light source is activated.

Blind and/or shield 406 can be movable with respect to body 404 housing a UV bulb, for example, to controllably deflect UV radiation emitted therefrom to select (e.g. target) specific regions 110 of facility 108. In some embodiments, blind and/or shield 406 can be movable with respect to body 404 housing a UV bulb by controller 104, for example, to controllably deflect UV radiation emitted therefrom to select (e.g. target) specific person-free regions 110 of facility 108 in response to receiving a signal indicating an absence of a person in region 110. In one embodiment, blind and/or shield 406 can retract from body 404 to deflect UV radiation emitted from the UV bulb of UV light source 400. In another embodiment, the blind and/or shield 406 can be unfurled from within a housing of body 404 to deflect UV radiation emitted from the UV bulb of UV light source 400.

Generally, controller 104 can control movement of the blind and/or shield 406 in response to determining a presence or an absence of a person in one of the plurality of regions 110 of the facility 108 based on the temperature data collected by sensors 102.

In another embodiment, UV light source can emit collimated light to control the emission of UV radiation within facility 108. Collimated light is generally light (including UV light) whose rays are parallel, and therefore will spread minimally as it propagates. Light can be approximately collimated by a number of processes, for instance by means of a collimator.

Herein, UV light source 106 may comprise a collimator to collimate UV radiation emitted therefrom in response to controller 104 determining a presence or an absence of a person in one of the plurality of regions 110 of the facility 108 based on the temperature data collected by sensors 102.

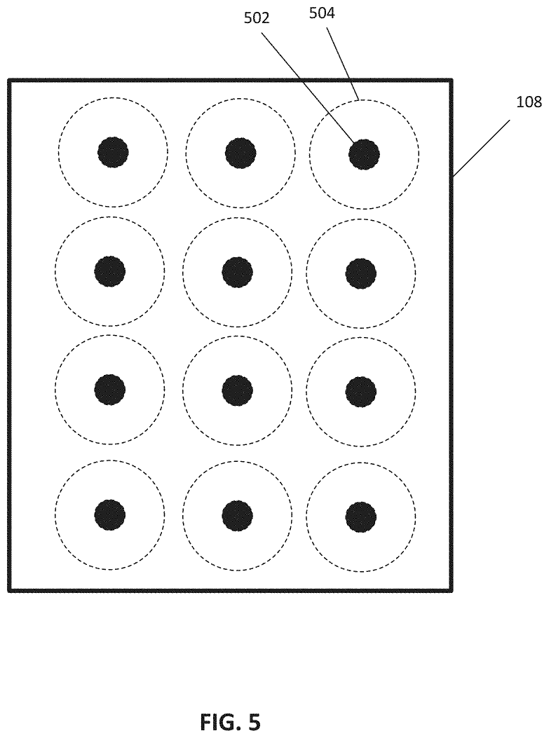

In another embodiment, shown in FIG. 5, light source 106 may be configured to be a UV potlight 502 (e.g. having a circular body) having an emitting area 504 as shown. Emitting area 504 is generally smaller than an emitting area of the example UV light source 106 shown in FIGS. 3 and 4, where the emitting area is approximately the same or larger than an area of facility 108.

A plurality of UV potlights 502 may be positioned in a facility 108 in a pattern such that their respective emitting areas 504 substantially cover the area of facility 108. In this embodiment, each respective emitting area 504 may correspond to a region 110 of sensor 102 (as shown in FIG. 2) and controller 104 may controllably activate individual UV potlights 502 in response to detecting an absence of a person in a respective region 110 of emitting area 504 of a UV potlight 502. In this manner, controller 104 may inhibit a person or persons from being exposed to UV radiation emitted by UV potlights 502. Accordingly, in this embodiment, a person or persons may be present in facility 108 during activation of potlights 502 and not be exposed to UV radiation emitted by potlights 502.

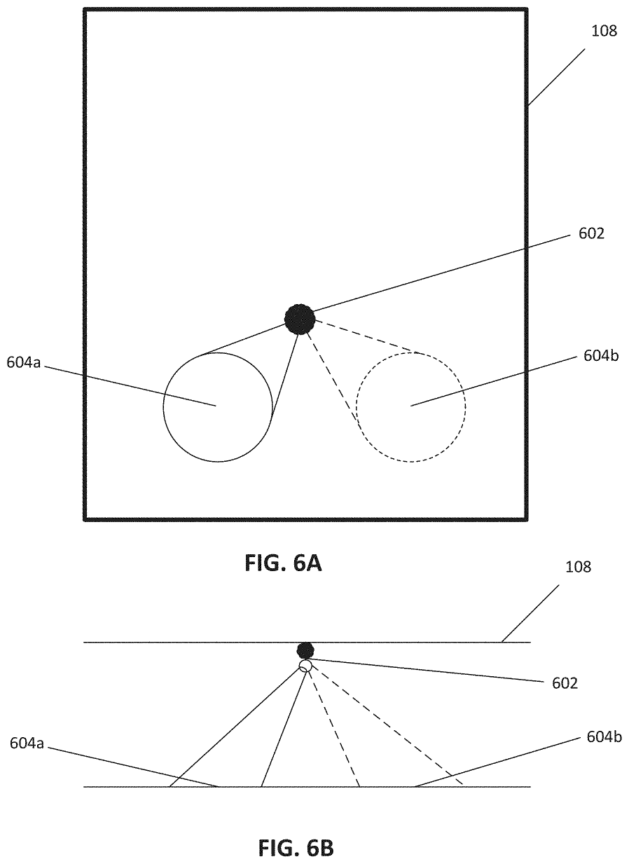

In another embodiment shown in FIGS. 6A and 6B, a UV light source 602 may have a defined (e.g. defined in size by using a collimator to narrow a beam of UV radiation) emitting area 604a and be movable (e.g. rotatable) about a point 606. In this embodiment, controller 104 may be used to move (e.g. rotate) UV light source 602 to shift emitting area 604a to a selectable (e.g. targeted) position (shown as emitting area 604b) within facility 108. In this manner, controller 104 may control light source 602 (e.g. in real-time) and emitting area 604a to, for example, follow-up a person as the person moves through facility 108 (e.g. between regions 110) to disinfect portions of the facility 108 while inhibiting the person in facility 108 from be exposed to UV radiation (e.g. to inhibit UV radiation from directly contacting a person or people present in facility 108) when the UV light source 602 is activated. Controller 104 may automatically disinfect one or more regions 110 upon receiving a signal indicating an absence of a person in a respective region 110 of the facility 108.

In use, each sensor 102 transmits a signal to the controller 104 over communication network 103. The signal may be periodically (e.g. at set time intervals) transmitted by each sensor 102 using the co-ordinates of the controller 104 in the facility 108 at that point in time or, alternatively, the signal may be transmitted upon sensing movement of a person within a region 110 (e.g. a change in the thermogram of the facility). Further, the thermographic data may be temporarily stored and periodically (e.g. at set time intervals or in response to a request for data from controller 104) transmitted by sensor 102 to controller 104 or, alternatively, the thermographic data may be transmitted by sensors 102 to controller 104 upon receipt by sensors 102 (e.g. upon sensors 102 sensing movement of a person within a region 110 of facility 108).

In one exemplary embodiment, facility 108 can represent a patient room in a hospital, or any other room in need of disinfection, and sensors 102 can be installed to detect the presence or absence of persons (e.g. patients, nurses, physicians, other health care professionals, etc.) within the room. In one specific embodiment, one or more sensors 102 can be installed on a ceiling of the room and used to detect the presence or absence of people in specific regions 110 and across specific regions 110 of the room. In this example, one or more sensors 102 can detect thermographic radiation emitted from persons in all areas of the patient room to determine the regions 110 of the room in which a person is present and the regions 110 of the room in which a person is absent. In other embodiments, sensors 102 may be mounted to other surfaces of a facility, such as but not limited to the walls or a floor, to determine the regions 110 of the room in which a person is present and the regions 110 of the room in which a person is absent.

In one embodiment, UV light source(s) 106 can be mounted to a ceiling in a patient room, or any other room in need of disinfection. In one specific embodiment, UV light source(s) 106 can be mounted to a ceiling such that an emitting area 404 of the UV light source(s) 106 substantially covers an area of the room 108. Under the control of controller 104, UV light source(s) 106 can be turned on/off and/or have components thereof (e.g. shields and/or blinds) controlled by the controller 104 to disinfect specific regions 110 of the room 108. In some embodiments, a person(s) may be present in at least one region 110 of the room 108 when other respect regions 110, where there is an absence of a person or persons, are disinfected with UV radiation from the UV light source(s).

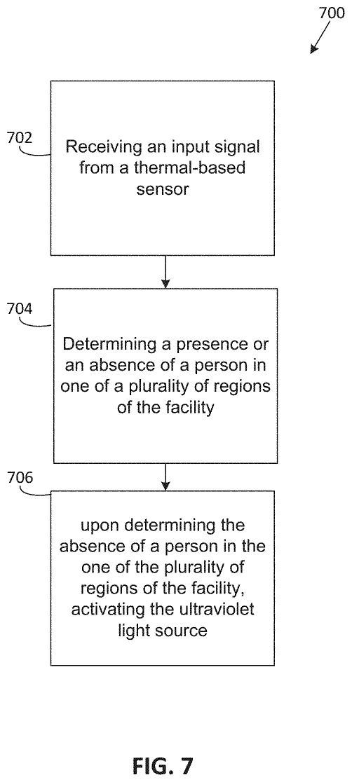

Turing to FIG. 7, illustrated therein is a method 700 of disinfecting a facility. At a first step 702, controller 104 receives an input signal from a thermal-based sensor 102, the input signal comprising temperature data based on thermal infrared radiation detected by the sensor 102 in the facility 108.

At a second step 704, the controller 104 determines a presence or an absence of a person in one of a plurality of regions 110 of the facility 108 based on the temperature data.

At a third step 706, upon determining the absence of a person in the one of the plurality of regions of the facility, the controller 104 activates the ultraviolet light source 106 to disinfect the one of the plurality of regions 110.

In at least one embodiment, the systems and methods described herein can use sensors other than thermal-based imaging sensors to detect and/or track the presence of individuals in the facility. For instance, other tracking or vision technologies such as but not limited to Laser Imaging Detection and Ranging (LIDAR) can be used to detect and/or track the presence of target (e.g. individuals) in the facility. In these embodiments, the controller of the systems described herein can receive data (e.g. object tracking data) from the sensors and the controller can determine the presence or the absence of a person in one or more regions of the facility based on the object tracking data. For instance, the controller may determine the presence or the absence of the person in one or more regions of the facility based on a positioning and/or movement of an object in the facility over time. Upon determining the absence of a person in the one of the plurality of regions of the facility, the controller then activates the ultraviolet light source to disinfect the one of the plurality of regions.

While the above description provides examples of one or more apparatus, methods, or systems, it will be appreciated that other apparatus, methods, or systems may be within the scope of the claims as interpreted by one of skill in the art.

What is claimed is:

1. A facility disinfection system, comprising:
a thermal-based imaging sensor configured to detect thermal infrared radiation in the facility, the facility having a plurality of regions;
an ultraviolet light source to provide ultraviolet radiation to disinfect the facility; and
a controller configured to:
receive an input signal from the thermal-based sensor, the input signal comprising temperature data based on the detected thermal infrared radiation in the facility;
determine a presence or an absence of a person in one of the plurality of regions of the facility based on the temperature data; and
upon determining the absence of a person in the one of the plurality of regions of the facility, activate the ultraviolet light source to disinfect the one of the plurality of regions.

2. The system of claim 1, wherein the controller is configured to upon determining the presence of a person in the one of the plurality of regions of the facility, control the ultraviolet light source to inhibit providing the ultraviolet radiation to the one of the plurality of regions, and activate the ultraviolet light source to disinfect a second portion of the facility portion of the facility upon determining an absence of the person in the portion of the facility.

3. The system of claim 1, wherein the ultraviolet light source is controllable and the controller controls the dissemination of UV radiation produced by the ultraviolet light source to a selected region of the plurality of regions.

4. The system of claim 3, wherein the ultraviolet light from the ultraviolet light source is collimated to control dissemination.

5. The system of claim 1, wherein the ultraviolet light source comprises a shield to deflect UV radiation produced by the ultraviolet light source.

6. The system of claim 5, wherein the controller controls movement of the shield to control the dissemination of UV radiation produced by the ultraviolet light source to a selected region of the plurality of regions.

7. The system of claim 1, wherein the ultraviolet light from the ultraviolet light source is rotatable.

8. A method of disinfecting a facility, the method comprising:
receiving an input signal from a thermal-based sensor, the input signal comprising temperature data based on thermal infrared radiation detected by the sensor in the facility;
determining a presence or an absence of a person in one of a plurality of regions of the facility based on the temperature data; and
upon determining the absence of a person in the one of the plurality of regions of the facility, activating the ultraviolet light source to disinfect the one of the plurality of regions.

9. The method of claim 8, wherein, upon determining the presence of a person in the one of the plurality of regions of the facility, the method further includes controlling the ultraviolet light source to inhibit providing the ultraviolet radiation to the one of the plurality of regions, and activating the ultraviolet light source to disinfect a second portion of the facility portion of the facility upon determining an absence of the person in the portion of the facility.

10. The method of claim 8, wherein the ultraviolet light source is controllable and the controller controls the dissemination of UV radiation produced by the ultraviolet light source to a selected region of the plurality of regions.

11. The method of claim 10, wherein the ultraviolet light from the ultraviolet light source is collimated to control dissemination.

12. The method of claim 8, wherein the ultraviolet light source comprises a shield to deflect UV radiation produced by the ultraviolet light source.

13. The method of claim 12, wherein the controller controls movement of the shield to control the dissemination of UV radiation produced by the ultraviolet light source to a selected region of the plurality of regions.

14. The method of claim 8, wherein the ultraviolet light from the ultraviolet light source is rotatable.

15. A facility disinfection system, comprising:
at least one sensor configured to detect an object in the facility, track a position of the object in the facility and output object tracking data based on the position of the object over time, the facility having a plurality of regions;
an ultraviolet light source to provide ultraviolet radiation to disinfect the facility; and
a controller configured to:
receive the signal from the at least one sensor, the signal comprising the object tracking data;
determine a presence or an absence of a person in one of the plurality of regions of the facility based on the object tracking data; and
upon determining the absence of a person in the one of the plurality of regions of the facility, activate the ultraviolet light source to disinfect the one of the plurality of regions.
